(19) United States
(12) Patent Application Publication
(10) Pub. No.: US 2025/0313558 A1
(43) Pub. Date: Oct. 9, 2025

(54) IMIDAZOLIDINE DERIVATIVE OR PHARMACEUTICALLY ACCEPTABLE SALT THEREOF, AND USE THEREOF

(71) Applicant: CELLUS INC., Daegu (KR)

(72) Inventors: Seong Keun KIM, Seoul (KR); Sung Moo KIM, Seongnam-si, Gyeonggi-do (KR); Sun Wook CHO, Seoul (KR); Min Joo KIM, Seoul (KR)

(21) Appl. No.: 18/863,238

(22) PCT Filed: Mar. 6, 2024
(86) PCT No.: PCT/KR2024/002865

§ 371 Date: Nov. 5, 2024

(30) Foreign Application Priority Data
Mar. 17, 2023 (KR) 10-2023-0035352

(51) Int. Cl. C07D 471/04; A61K 31/437; A61P 19/02

(52) U.S. Cl. CPC C07D 471/04; A61K 31/437; A61P 19/02

(57) ABSTRACT

Embodiments relate to an imidazolidine derivative or a pharmaceutically acceptable salt thereof and its use for preventing, ameliorating, or treating arthritis. The compound of one embodiment can be effectively utilized for preventing, ameliorating, or treating arthritis by inhibiting degeneration or damage to cartilage tissue through inhibition of the activity of ADAMTS4 or ADAMTS5.

### TECHNICAL FIELD

The present invention relates to an imidazolidine derivative or a pharmaceutically acceptable salt thereof and its use for preventing, ameliorating, or treating arthritis.

### BACKGROUND ART

In modern society, as the population ages, the number of patients with degenerative diseases (e.g., dementia, arthritis, and arteriosclerosis) is increasing, and the subsequent hindrance of social activities of the elderly people brings economic and mental hardships to their families as well as to themselves, thus becoming a social problem.

Recently, as Korea has entered an aging society, it is known that 80% of people aged 55 or more and almost all people aged 75 or more have osteoarthritis. In particular, knee osteoarthritis has a very high incidence rate, and when inflammation continues, it can cause atrophy of the surrounding muscles thereby even causing joint deformities. Therefore, osteoarthritis is a disease that causes significant social losses including treatment costs, hindrance of social activities, etc. of the elderly population.

Osteoarthritis (degenerative arthritis) is a common geriatric disease observed in clinical practice due to the aging population, in which lesions begin based on degenerative changes in articular cartilage, causing pain, swelling, joint stiffness, and progressive motor impairment during exercise.

The pathological causes of degenerative arthritis known to date are known to be caused by biochemical changes in articular cartilage due to mechanical damage to the joint, genetic factors, obesity, etc. These biochemical changes that lead to degenerative arthritis are due to the degradation of the cartilage matrix by increased expression and activation of matrix metalloproteinases (MMPs) and a disintegrin and metalloproteinase with thrombospondin motifs (ADAMTS), which are cartilage matrix degrading enzymes, and decreased synthesis of cartilage matrix molecules by death and loss of characteristics (dedifferentiation) of chondrocytes. MMPs and ADAMTS produced in chondrocytes degrade various ECM (extracellular matrix) molecules, such as collagen and proteoglycan, synthesized and secreted from chondrocytes, resulting in the destruction of cartilage tissue and changes in the composition of cartilage tissue ECM molecules. MMPs and ADAMTS produced in chondrocytes degrade various extracellular matrix (ECM) molecules synthesized and secreted from chondrocytes (e.g., collagen, proteoglycan, etc.), thereby ultimately destroying cartilage tissues and changing the constitution of cartilage tissue ECM molecules.

Therefore, the regulation of synthesis and activity of MMPs and ADAMTS is becoming an important target for controlling cartilage degeneration, but there is still a need for the development of substances that can effectively inhibit the same.

### DISCLOSURE OF INVENTION

### Technical Problem

An object to be achieved by the present invention is to provide an imidazolidine derivative or a pharmaceutically acceptable salt thereof.

Another object to be achieved by the present invention is to provide a pharmaceutical composition or a health functional food for preventing or treating arthritis.

The objects to be achieved by the present invention are not limited to the those described above, and other objects not described above can be clearly understood by those having ordinary skill in the art to which the present invention belongs from the description below.

### Technical Solution

In order to achieve the above-identified objects, an embodiment of the present invention provides an imidazolidine derivative represented by the following Formula 1 or a pharmaceutically acceptable salt thereof.

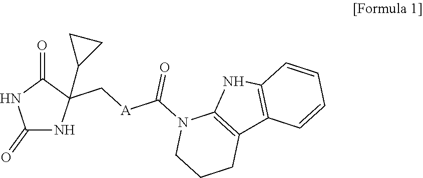

(wherein A is a C₁₋₃ alkyl group or amine group)

In an embodiment of the present invention, the imidazolidine derivative may be a compound represented by the following Formula 2 or Formula 3.

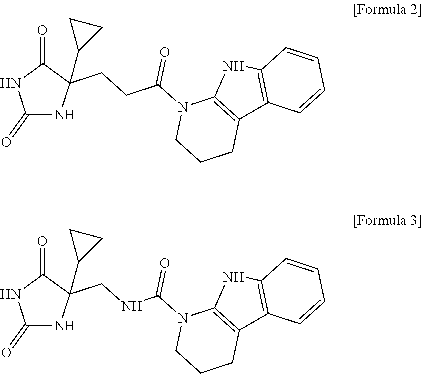 [SMILES: O=C1NC(=O)C(CCC(=O)N2CCCc3c2[nH]c2ccccc32)(C2CC2)N1 ; O=C1NC(=O)C(CNC(=O)N2CCCc3c2[nH]c2ccccc32)(C2CC2)N1]

In order to achieve the above-identified objects, another embodiment of the present invention provides a pharmaceutical composition for preventing or treating arthritis, which includes an imidazolidine derivative represented by Formula 1 above or a pharmaceutically acceptable salt thereof.

In order to achieve the above-identified objects, another embodiment of the present invention provides a health functional food for preventing or ameliorating arthritis, which includes an imidazolidine derivative represented by Formula 1 above or a sitologically acceptable salt thereof.

In an embodiment of the present invention, the prevention, amelioration, or treatment may be achieved by inhibiting the activity of a disintegrin and metalloproteinase with thrombospondin motifs 4 (ADAMTS4) or a disintegrin and metalloproteinase with thrombospondin motifs 5 (ADAMTS5).

In an embodiment of the present invention, the arthritis may be osteoarthritis, degenerative arthritis, rheumatoid arthritis, avascular necrosis of the femoral head, traumatic arthritis, or tuberculous septic arthritis.

### Advantageous Effects

The present invention relates to an imidazolidine derivative or a pharmaceutically acceptable salt thereof, and a use thereof in preventing, ameliorating, or treating arthritis. A compound according to the present invention inhibits degeneration or damage to cartilage tissue by inhibiting the activity of ADAMTS4 or ADAMTS5 can thus be effectively used in preventing, ameliorating, or treating arthritis.

The effects of the present invention are not limited to those described above, and should be understood to include all effects that can be inferred from the constitutions of the invention described in the description or claims of the present invention.

### BRIEF DESCRIPTION OF THE DRAWINGS

FIGS. 1 to 4 show the comparison results of inhibition abilities of five compounds, at different concentrations, against ADAMTS5, ADAMTS4, ADAM10, and ADAM17, respectively.

FIG. 5 show the comparison results of IC₅₀ values of seven compounds for inhibition of ADAMTS5, ADAMTS4, ADAM10, and ADAM17.

FIGS. 6 and 7 show the evaluation results of cell growth and toxicity performed 24 and 48 hours after treating chondrocytes with five compounds, respectively.

FIGS. 8 and 9 show the evaluation results of ex vivo efficacy on cartilage tissue degeneration, in which FIG. 9 confirms the reduction in cartilage damage through Alcian blue staining, and FIG. 10 analyzes GAG release through DMMB assay.

FIG. 10 shows the results of confirming the effect of effective substances (CLS-S210, CLS-S282) according to an embodiment of the present invention on reducing cartilage damage and pain.

### BEST MODE FOR CARRYING OUT THE INVENTION

Hereinafter, the present invention will be described in detail.

The present invention relates to an imidazolidine derivative represented by the following Formula 1 or a pharmaceutically acceptable salt thereof.

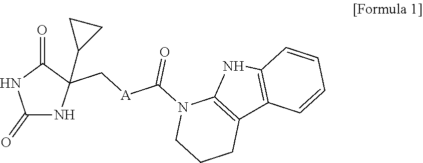

(wherein A is a C₁₋₃ alkyl group or amine group)

As used herein, the term “pharmaceutically acceptable” intends to refer to compounds, substances, compositions and/or dosage forms that are suitable for use in contact with human tissues, without excessive toxicity, irritation, allergic response, or other problems or complications, within the scope of sound medical judgment, and complies with a reasonable benefit/risk ratio.

The “pharmaceutically acceptable salt” refers to a derivative of the disclosed compound, wherein the parent compound is modified by preparing its acid or base salt. Examples thereof may include inorganic or organic acid salts of basic residues such as amines; alkali or organic salts of acidic residues such as carboxylic acids; etc., but are not limited thereto. For example, it may include conventional non-toxic salts or quaternary ammonium salts of the parent compound formed from non-toxic inorganic or organic acids. For example, such common non-toxic salts may include salts derived from inorganic acids, such as hydrochloric acid, hydrobromic acid, sulfuric acid, sulfamic acid, phosphoric acid, nitric acid, etc.; and salts derived from organic acids, such as acetic acid, propionic acid, succinic acid, glycolic acid, stearic acid, lactic acid, malic acid, tartaric acid, citric acid, ascorbic acid, pamoic acid, maleic acid, hydroxymaleic acid, phenylacetic acid, glutamic acid, benzoic acid, salicylic acid, sulfanilic acid, 2-acetoxybenzoic acid, fumaric acid, toluenesulfonic acid, methanesulfonic acid, ethane disulfonic acid, oxalic acid, isethionic acid, etc.

Pharmaceutically acceptable salts of a compound may be synthesized from a parent compound containing a basic or acidic moiety by conventional chemical methods. In general, these salts may be prepared by reacting the free acid or base form of the compound with a stoichiometric amount of the appropriate base or acid in water or an organic solvent or a mixture thereof. Generally, non-aqueous media such as ether, ethyl acetate, ethanol, isopropanol, or acetonitrile are preferred. A list of suitable salts is disclosed in Remington's Pharmaceutical Sciences, 17th ed., Mack Publishing Company, Easton, PA, 1985, p. 1418, and the disclosure of which is incorporated herein by reference in its entirety.

Additionally, the present invention includes not only pharmaceutically acceptable salts of the imidazolidine derivatives, but also all possible solvates and hydrates that can be prepared therefrom, and may also include all possible stereoisomers.

The imidazolidine derivative may be a compound represented by the following Formula 2 or Formula 3.

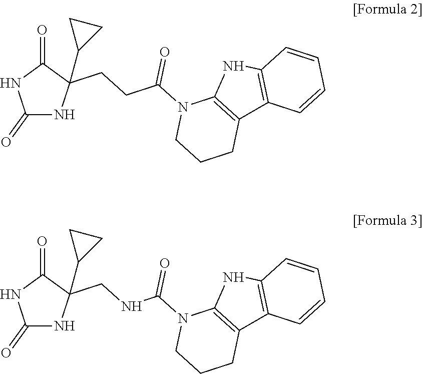 [SMILES: O=C1NC(=O)C(CCC(=O)N2CCCc3c2[nH]c2ccccc32)(C2CC2)N1 ; O=C1NC(=O)C(CNC(=O)N2CCCc3c2[nH]c2ccccc32)(C2CC2)N1]

The present invention relates to a pharmaceutical composition for preventing or treating arthritis.

The pharmaceutical composition of the present invention includes an imidazolidine derivative represented by Formula 1 above or a pharmaceutically acceptable salt thereof.

The imidazolidine derivatives and pharmaceutically acceptable salts thereof are as described above.

The arthritis is a chronic degenerative joint disease that causes pain, joint destruction, and loss of function due to inflammatory damage to the articular cartilage, and examples may include osteoarthritis, degenerative arthritis, rheumatoid arthritis, avascular necrosis of the femoral head, traumatic arthritis, or tuberculosis septic arthritis, but are not limited thereto.

The prevention or treatment may be, for example, achieved by inhibition of the activity of a disintegrin and metalloproteinase with thrombospondin motifs 4 (ADAMTS4) or a disintegrin and metalloproteinase with thrombospondin motifs 5 (ADAMTS5). That is, the compound of the present invention acts specifically on ADAMTS4 and ADAMTS5 and inhibits their activity, and thus inhibits cartilage matrix decomposition or protecting cartilage tissue, thereby having an excellent effect on prevention or treatment of arthritis.

The pharmaceutical composition of the present invention further includes a pharmaceutically acceptable carrier, which is commonly used in formulations. The pharmaceutically acceptable carrier, being commonly used in formulations, includes lactose, dextrose, sucrose, sorbitol, mannitol, starch, acacia gum, calcium phosphate, alginate, gelatin, calcium silicate, microcrystalline cellulose, polyvinylpyrrolidone, cellulose, water, syrup, methyl cellulose, methyl hydroxybenzoate, propyl hydroxybenzoate, talc, magnesium stearate, mineral oil, etc., but is not limited thereto.

The pharmaceutical composition of the present invention may additionally include a lubricant, a wetting agent, a sweetener, a flavoring agent, an emulsifier, a suspending agent, a preservative, etc., in addition to the above-described ingredients. Suitable pharmaceutically acceptable carriers and formulations are described in detail in Remington's Pharmaceutical Sciences (19th ed., 1995). The appropriate dose of the pharmaceutical composition of the present invention varies depending on factors, such as the formulation method, administration method, patient's age, weight, sex, degree of disease symptoms, food, administration time, administration route, excretion rate, and response sensitivity. Typically, a skilled physician can easily determine and prescribe an effective dose for the intended treatment. Meanwhile, the dose of the pharmaceutical composition of the present invention may be 0.01-2,000 mg/kg (body weight) per day, but is not limited thereto.

The pharmaceutical composition of the present invention may be administered orally or parenterally, and when administered parenterally, it may be administered by intravenous injection, subcutaneous injection, intramuscular injection, intraperitoneal injection, transdermal administration, etc. It is preferable that the administration route of the pharmaceutical composition of the present invention be determined depending on the type of disease to which it is applied.

The pharmaceutical composition of the present invention may be prepared in a unit dose form or may be prepared by placing it into a multi-dose container by formulating it using a pharmaceutically acceptable carrier and/or excipient according to a method that can easily be performed by a person having ordinary skill in the art to which the present invention pertains. In particular, the formulation may be in the form of a solution, suspension, or emulsion in an oil or aqueous medium, or in the form of an extract, powder, granules, tablet, or capsule, and may additionally include a dispersant or stabilizer.

The present invention relates to a health functional food for preventing or improving arthritis.

The health functional food of the present invention includes an imidazolidine derivative represented by Formula 1 above or a sitologically acceptable salt thereof.

The compounds included in the health functional food of the present invention, their mechanism of action, and the types of arthritis are as described above.

The health functional food of the present invention may be prepared and processed in the form of tablets, capsules, powders, granules, liquids, pills, etc. for the purpose of preventing or improving arthritis.

The health functional food of the present invention refers to a food prepared and processed using raw materials or ingredients that have functionality useful to the human body, and is meant to be consumed for the purpose of obtaining useful effects for health purposes such as regulating nutrients with respect to the structure and function of the human body or physiological effects.

The health functional food of the present invention may include conventional food additives, and the suitability as food additives is determined by the standards and criteria for the relevant items according to the general provisions and general test methods of the Food Additive Code approved by the Ministry of Food and Drug Safety of Korea, unless otherwise specified.

The items listed in the food additive code include chemical compounds (e.g., ketones, glycine, calcium citrate, nicotinic acid, cinnamic acid, etc.); natural additives (e.g., citrus pigments, crystalline cellulose, high-molecular-weight pigments, guar gum, etc.); and mixed preparations (e.g., sodium L-glutamate preparations, noodle-added alkaline preparations, preservative preparations, tar color preparations, etc., but are not limited thereto.

For example, a health functional food in tablet form may be prepared by mixing the compound with an excipient, a binder, a disintegrant, and other additives; granulating the mixture in a conventional manner; adding a lubricant, etc.; and compressing the resultant, or may be prepared by directly compressing the mixture. In addition, the health functional food in tablet form may include a flavor enhancer, etc., as necessary.

Among health functional foods in capsule form, hard capsules may be prepared by filling a mixture of the compound and additives (e.g., excipients, etc.) into a normal hard capsule, and soft capsules may be prepared by filling a mixture of the compound and additives (e.g., excipients, etc.) into a capsule base such as gelatin. The soft capsule may include a plasticizer (e.g., glycerin or sorbitol), a coloring agent, a preservative, etc., as necessary.

A health functional food in the form of a ring may be prepared by mixing the compound and an excipient, a binder, a disintegrant, etc., and molding the mixture using a conventionally known method. If necessary, the mixture may be coated with white sugar or a different coating agent, or the surface may be coated with a substance such as starch or talc.

Granular health functional foods may be prepared into granules by mixing the compound and an excipient, a binder, a disintegrant, etc. using a conventionally known method, and may include a flavoring agent, a flavor enhancer, etc., as necessary.

The health functional foods may include beverages, meats, chocolate, foods, confectioneries, pizza, ramen, other noodles, gums, candies, ice creams, alcoholic beverages, vitamin complexes, health supplements, etc.

The health functional food may be orally applied as a nutritional supplement, and the form of application is not particularly limited.

Hereinafter, the present invention will be described in detail by way of examples in order to specifically describe the present invention.

### Experimental Method and Results

### 1. Confirmation of Inhibition Ability of CLS-S2 Compound Against ADAM/ADAMTS Proteins Through FRET Assay

The control compound GLPG-1972 and four effective compounds (CLS-S210 (Formula 2), CLS-S282 (Formula 3), CLS-S213 (Formula 4), and CLS-S214 (Formula 5)) were treated with ADAMTS5, ADAMTS4, ADAM10, and ADAM17 proteins to perform Fluorescence Resonance Energy Transfer (FRET) evaluation. FRET assay is a method to detect and quantify the fluorescent signal generated when ADAM/ADAMTS proteins deliver specific peptides.

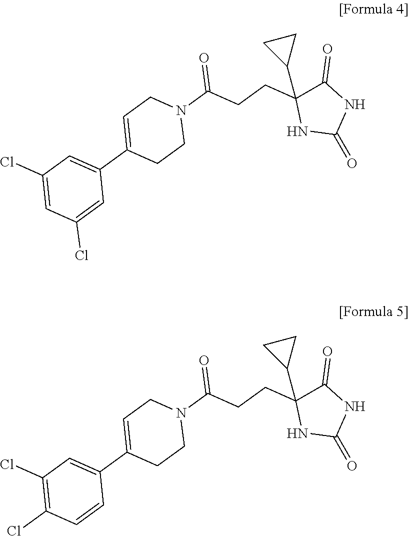 [SMILES: O=C1NC(=O)C(CCC(=O)N2CC=C(c3cc(Cl)cc(Cl)c3)CC2)(C2CC2)N1 ; O=C1NC(=O)C(CCC(=O)N2CC=C(c3ccc(Cl)c(Cl)c3)CC2)(C2CC2)N1]

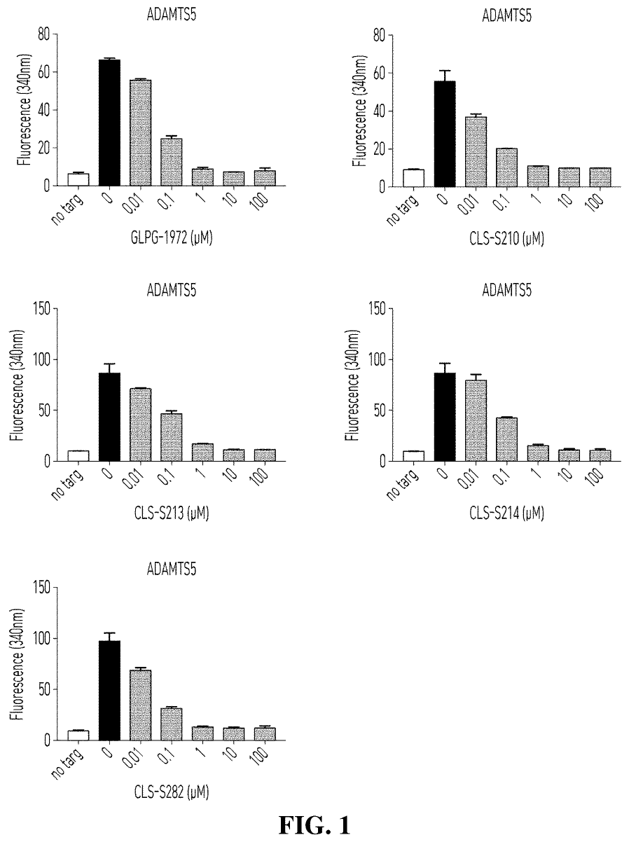

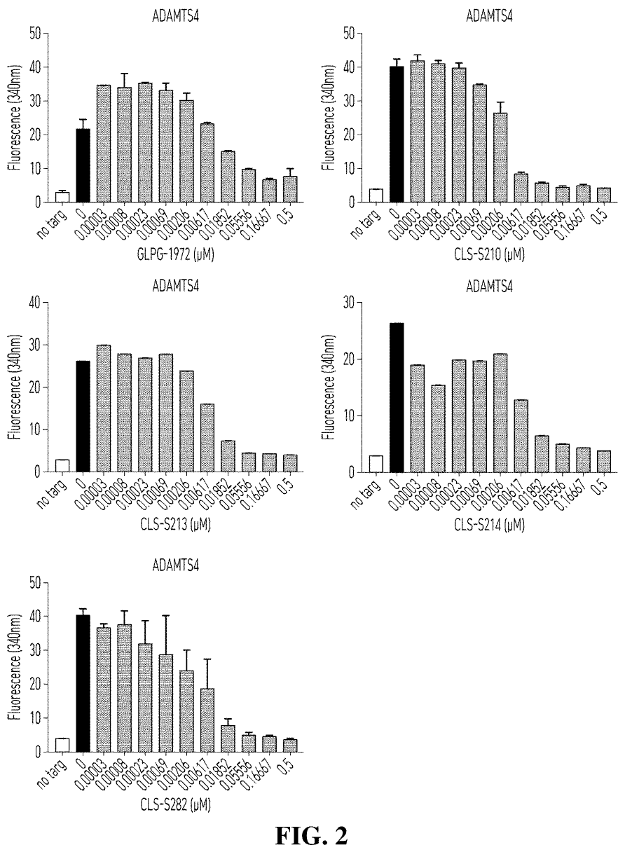

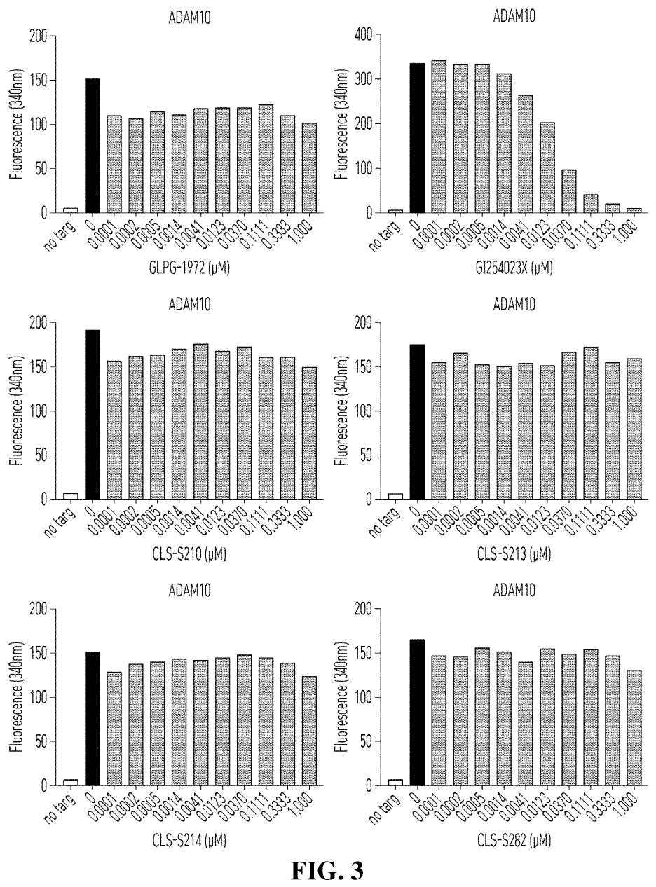

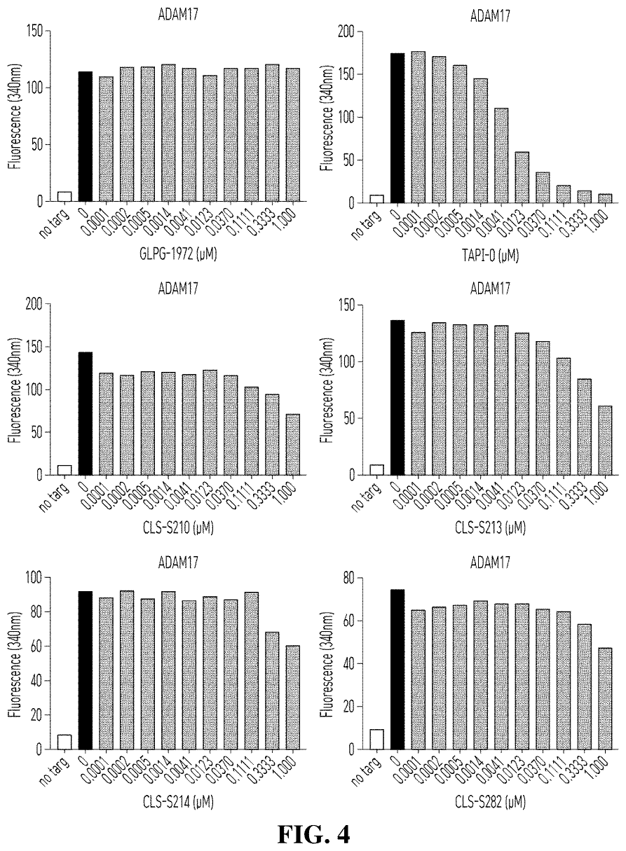

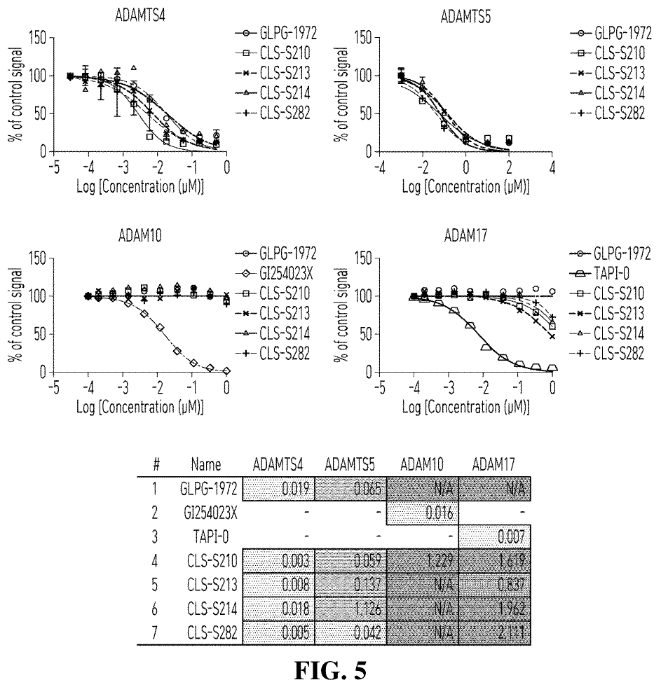

CLS-S210 (IC₅₀=0.059 μM) and CLS-S282 (IC₅₀=0.042 μM) compounds were confirmed to have superior inhibition ability against ADAMTS5 compared to the control compound (IC₅₀=0.065 μM) (FIGS. 1 and 5). Additionally, the five compounds were confirmed to effectively inhibit not only ADAMTS5 but also ADAMTS4 at low concentrations (FIGS. 2 and 5). The four effective compounds did not show significant inhibition ability against ADAM10 and ADAM17 (FIGS. 3, 4, and 5). These results suggest that these compounds act specifically on ADAMTS4 and ADAMTS5.

In addition, an additional FRET assay was performed on ADAM10 and ADAM17, which were not effectively inhibited by the four effective CLS-S2 compounds. GI204023X is known as an ADAM10 inhibitor, and TAPI-0 is known to effectively inhibit ADAM17. As a result of the experiment, GI254023X and TAPI-0 were shown to inhibit ADAM10 and ADAM17 at low concentrations as is known, and thus, it was determined that there was no abnormality in the FRET system (FIG. 5).

### 2. Evaluation of Toxicity of CLS-S2 Compound on Chondrocytes

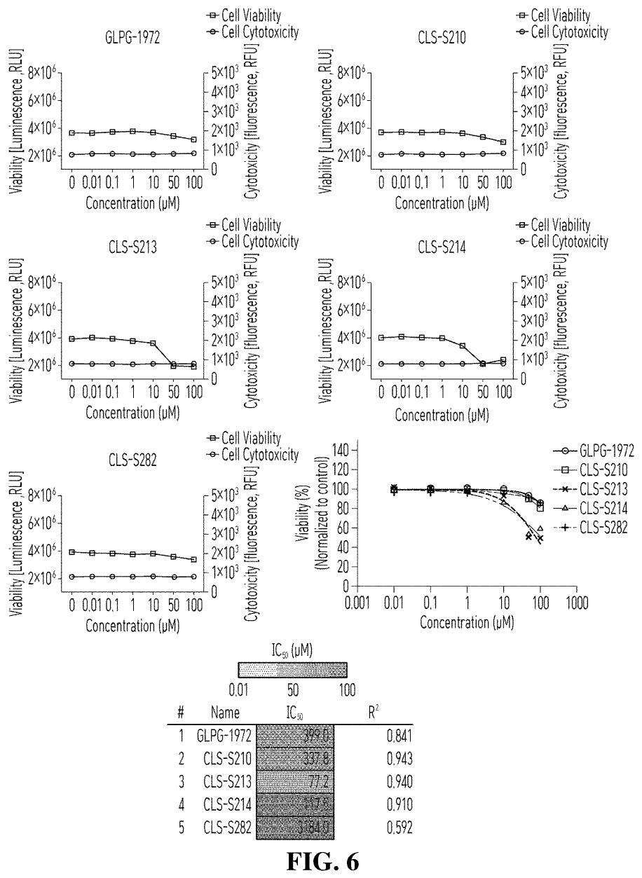

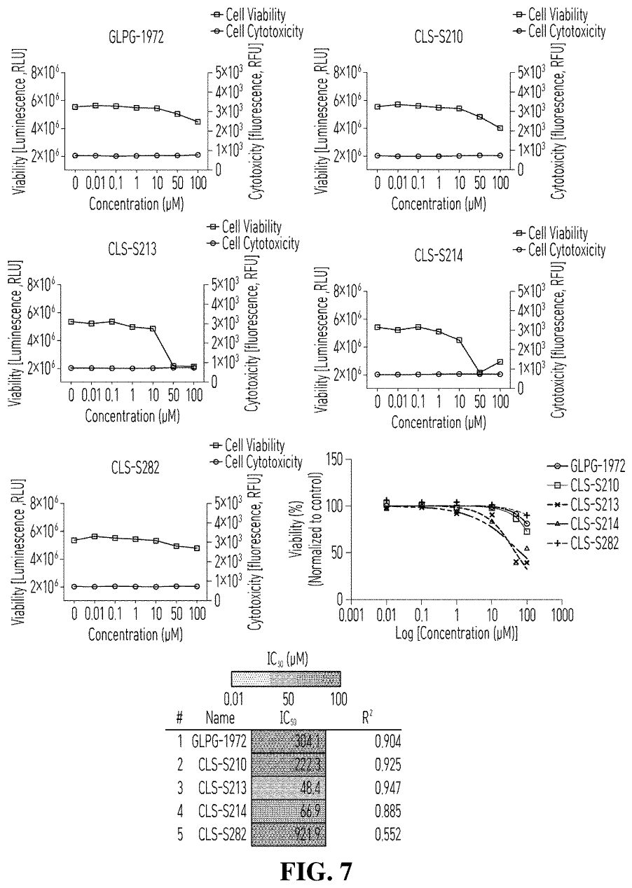

Chondrocytes were isolated from 3-day-old Sprague-Dawley (SD) rats, and GLPG-1972 (control compound) and four effective compounds were treated to the chondrocytes for 24 and 48 hours to evaluate changes in cell growth and toxicity. Compounds CLS-S210 and CLS-S282, which showed excellent inhibition ability against ADAMTS4 and ADAMTS5 in the FRET assay, showed lower chondrocyte toxicity compared to other compounds (FIGS. 6 and 7).

### 3. Ex Vivo Study Using Mouse Cartilage Explant

### (1) Visual Analysis of Cartilage Damage Using Alcian Blue Staining

Femoral heads of 5-day-old mouse were obtained and treated with each of ADAMTS5 targeting substances while simultaneously treating with IL-1β, and cultured for 72 hours. After culture, the culture medium was collected to confirm GAG release, and the cartilage tissue was fixed, made into a paraffin block, and sectioned using a microtome.

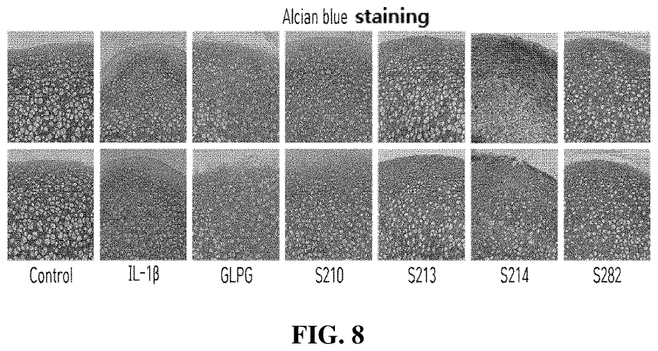

When each sectioned slide was stained with Alcian blue to determine the depth or area of the discolored area, it was confirmed that cartilage damage occurred when treated with IL-1β, whereas cartilage damage was reduced when treated with ADAMTS5 targeting effective substance (FIG. 8).

### (2) Analysis of GAG Release Using DMMB Assay

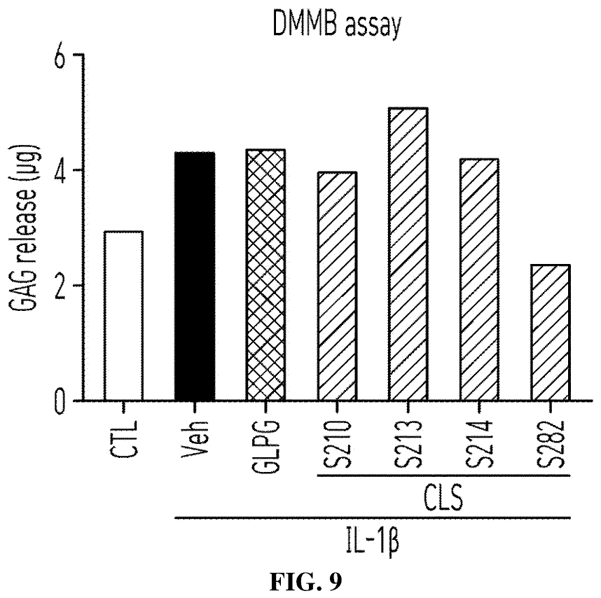

DMMB assay was performed to analyze the amount of cartilage matrix released by decomposition in the culture medium, and as a result, the amount of GAG measured in CLS-S210 and CLS-S282 among ADAMTS5 targeting effective substances was low, and it was confirmed that the compound has an excellent effect in protecting cartilage by inhibiting cartilage matrix decomposition (FIG. 9).

### 5. Evaluation of Therapeutic Efficacy in Mouse Model of Osteoarthritis (Osteoarthritis Regulation Mechanism of ADAMTS5 Targeting Effective Substances)

Osteoarthritis was artificially induced by surgically resecting the meniscus in the knee joint of a mouse, ADAMTS5 targeting substances were injected into the knee.

In order to observe the effect of each compound on cartilage degeneration in vivo, Safranin-O staining was performed and the OARSI grade, which can evaluate cartilage damage caused by osteoarthritis, was quantified.

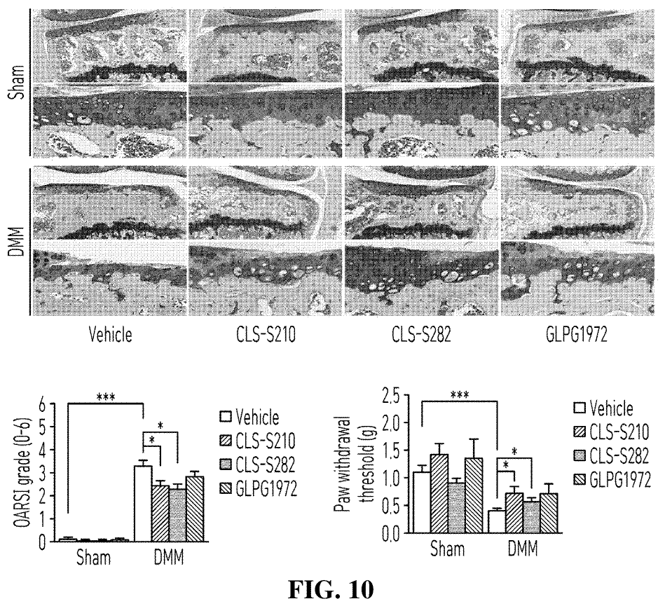

The degree of cartilage damage was evaluated by giving a score from 0 to 6 points. As a result of confirming the cartilage protection effect of ADAMTS5 targeting effective substances in mice of osteoarthritis induced through DMM surgery, in the case of osteoarthritis mice treated with vehicle, surface erosion and cartilage loss were clearly observed as characteristics, whereas in ADAMTS5 targeting compounds, cartilage damage was reduced, and a significant reduction was observed particularly in CLS-S210 and CLS-S282 compounds (FIG. 10).

As with the degree of cartilage damage, the pain sensitization experiment also showed a significant pain reduction by CLS-S210 and CLS-S282, which are ADAMTS5 targeting substances (FIG. 10).

1. An imidazolidine derivative represented by the following Formula 1 or a pharmaceutically acceptable salt thereof: 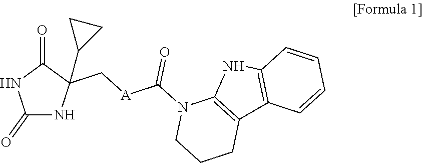
(wherein A is a C₁₋₃ alkyl group or amine group).

2. The imidazolidine derivative of claim 1, wherein the imidazolidine derivative is a compound represented by the following Formula 2 or Formula 3:

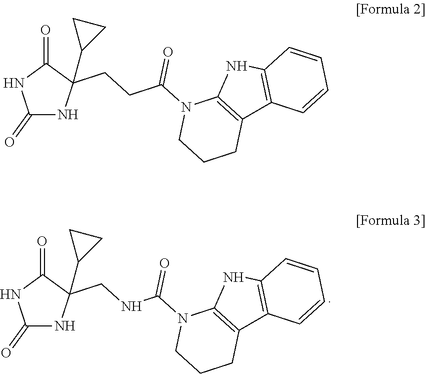 [SMILES: O=C1NC(=O)C(CCC(=O)N2CCCc3c2[nH]c2ccccc32)(C2CC2)N1 ; O=C1NC(=O)C(CNC(=O)N2CCCc3c2[nH]c2ccccc32)(C2CC2)N1]

3. A pharmaceutical composition for preventing or treating arthritis comprising an imidazolidine derivative represented by the following formula 1 or a pharmaceutically acceptable salt thereof: 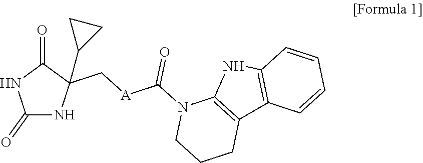
(wherein A is a C₁₋₃ alkyl group or amine group).

4. The pharmaceutical composition of claim 3, wherein the imidazolidine derivative is a compound represented by the following Formula 2 or Formula 3:

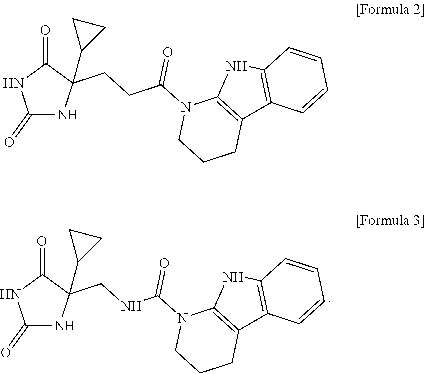 [SMILES: O=C1NC(=O)C(CCC(=O)N2CCCc3c2[nH]c2ccccc32)(C2CC2)N1 ; O=C1NC(=O)C(CNC(=O)N2CCCc3c2[nH]c2ccccc32)(C2CC2)N1]

5. The pharmaceutical composition of claim 3, wherein the arthritis is osteoarthritis, degenerative arthritis, rheumatoid arthritis, avascular necrosis of the femoral head, traumatic arthritis, or tuberculous septic arthritis.

6. The pharmaceutical composition of claim 3, wherein the prevention or treatment is achieved by inhibiting the activity of a disintegrin and metalloproteinase with thrombospondin motifs 4 (ADAMTS4) or a disintegrin and metalloproteinase with thrombospondin motifs 5 (ADAMTS5).

7. A health functional food for preventing or ameliorating arthritis, comprising an imidazolidine derivative represented by the following Formula 1 or a sitologically acceptable salt: 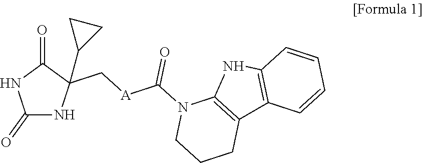
(wherein A is a C₁₋₃ alkyl group or amine group).

8. The health functional food of claim 7, wherein the imidazolidine derivative is a compound represented by the following Formula 2 or Formula 3:

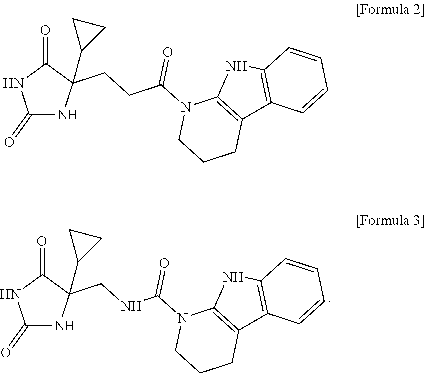 [SMILES: O=C1NC(=O)C(CCC(=O)N2CCCc3c2[nH]c2ccccc32)(C2CC2)N1 ; O=C1NC(=O)C(CNC(=O)N2CCCc3c2[nH]c2ccccc32)(C2CC2)N1]

9. The health functional food of claim 7, wherein the arthritis is osteoarthritis, degenerative arthritis, rheumatoid arthritis, avascular necrosis of the femoral head, traumatic arthritis, or tuberculous septic arthritis.

10. The health functional food of claim 7, wherein the prevention or treatment is achieved by inhibiting the activity of a disintegrin and metalloproteinase with thrombospondin motifs 4 (ADAMTS4) or a disintegrin and metalloproteinase with thrombospondin motifs 5 (ADAMTS5).

11. A method of treating arthritis, the method comprising administering to a patient in need of treatment an imidazolidine derivative represented by the following formula 1 or a pharmaceutically acceptable salt thereof: 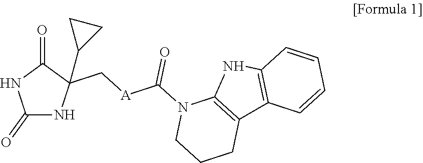
(wherein A is a C₁₋₃ alkyl group or amine group).

12. The method of claim 11, wherein the imidazolidine derivative is a compound represented by the following Formula 2 or Formula 3:

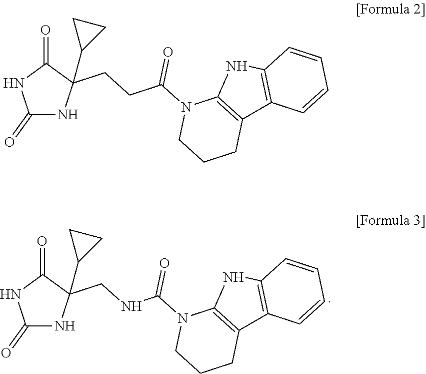 [SMILES: O=C1NC(=O)C(CCC(=O)N2CCCc3c2[nH]c2ccccc32)(C2CC2)N1 ; O=C1NC(=O)C(CNC(=O)N2CCCc3c2[nH]c2ccccc32)(C2CC2)N1]

13. The method of claim 11, wherein the arthritis is osteoarthritis, degenerative arthritis, rheumatoid arthritis, avascular necrosis of the femoral head, traumatic arthritis, or tuberculous septic arthritis.

14. The method of claim 11, wherein the imidazolidine derivative inhibits the activity of a disintegrin and metalloproteinase with thrombospondin motifs 4 (ADAMTS4) or a disintegrin and metalloproteinase with thrombospondin motifs 5 (ADAMTS5).
